(19) United States
(12) Patent Application Publication
(10) Pub. No.: US 2010/0324124 A1
(43) Pub. Date: Dec. 23, 2010

(54) Compositions and methods relating to DNA-based particles

(75) Inventors: Darrell J. Irvine, Arlington, MA (US); Soong Ho Um, Gwangsan-gu (KR); Dan Luo, Ithaca, NY (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 12/456,587

(22) Filed: Jun. 17, 2009

(51) Int. Cl. A61K 48/00; C12P 19/34; C07H 21/00

(52) U.S. Cl. 514/44 R; 435/91.52; 536/22.1; 428/402

(57) ABSTRACT

The invention provides compositions and methods relating to delivery of agents in vivo or in vitro. More specifically, the invention provides nanoparticles synthesized from crosslinked nucleic acids, optionally having a lipid shell or coating, and may further comprise for example small molecule or high molecular weight compounds as therapeutic or diagnostic agents.

### FEDERALLY SPONSORED RESEARCH

This invention was made with Government support under grant number DMR-0819762 from the National Science Foundation (NSF MRSEC). The Government has certain rights to this invention.

### BACKGROUND OF INVENTION

In vivo drug delivery approaches to date have focused in part on liposome-mediated delivery and biodegradable polymeric particles. Liposomes are the prototypical nanoscale drug carrier and have a variety of favorable properties, such as biocompatibility and biodegradability and an ability for sustained circulation times in the blood. However, liposomes are also known to be unstable in the presence of serum, often encapsulate only low levels of hydrophilic drugs, and have a limited ability to regulate the release of hydrophobic compounds. Biodegradable polymeric nanoparticles have been pursued as an alternative, but these synthetic particles also encapsulate relatively low levels of proteins or hydrophilic drugs and tend to have lower blood circulation times than liposomes. Polymeric nanoparticles also typically require the use of toxic organic solvents in their synthesis, which complicate translation to clinically acceptable formulations.

### SUMMARY OF INVENTION

The invention relates broadly to the delivery, including sustained delivery, of agents such as therapeutic and diagnostic (e.g., imaging) agents in vivo and in vitro. More specifically, the invention provides nanoparticles made from crosslinked nucleic acids comprising the agent(s) of interest. These nanoparticles are non-toxic owing to the nucleic acid matrix at their core and to the absence of organic solvents required in their production. The nanoparticles have the flexibility to entrap small molecules and/or high molecular weight proteins, and perhaps more importantly have demonstrated significantly extended release profiles. The methods for obtaining the nanoparticles of the invention were not known nor where they predictable to those of ordinary skill in the art.

Thus, in one aspect the invention provides a method comprising combining in solution branched nucleic acids, nucleic acid ligase, ATP, and lipids to form a mixture comprising lipid-encapsulated branched nucleic acids and free branched nucleic acids, incubating the mixture under conditions and for a time sufficient for the nucleic acid ligase to crosslink the branched nucleic acids, and harvesting crosslinked branched nucleic acids.

In some embodiments, the lipids are non-cationic phospholipids.

In some embodiments, the method further comprises removing the free branched nucleic acids from the mixture. In some embodiments, the harvested crosslinked branched nucleic acids are lipid-encapsulated. In some embodiments, the method further comprises removing lipids from the mixture prior to harvesting crosslinked branched nucleic acids. In some embodiments, the harvested crosslinked branched nucleic acids do not have a lipid coating.

In some embodiments, the method further comprises size selecting the lipid-encapsulated branched nucleic acids. In some embodiments, the method further comprises size selecting the crosslinked branched nucleic acids before or after harvest.

In some embodiments, the free branched nucleic acids are removed from the mixture using a nuclease. In some embodiments, nuclease is exonuclease.

In some embodiments, the lipids are removed using detergent or an enzyme. In some embodiments, the detergent is Triton-X. In some embodiments, the enzyme is a lipase such as a phospholipase.

In some embodiments, the branched nucleic acids are branched DNA. In some embodiments, the branched nucleic acids are X-shaped nucleic acids such as X-shaped DNA, or Y-shaped nucleic acids such as Y-shaped DNA, or T-shaped nucleic acids such as T-shaped DNA, or dendrimeric nucleic acids such as dendrimeric DNA, and the like.

In some embodiments, the branched nucleic acids are heterogeneous. In some embodiments, the branched nucleic acids are homogeneous. In some embodiments, the branched nucleic acids comprise branched nucleic acids having two crosslinking ends. In some embodiments, the branched nucleic acids comprise branched nucleic acids having three or more crosslinking ends.

In some embodiments, one or more therapeutic agents are combined with the branched nucleic acids prior to crosslinking, and the resulting crosslinked branched nucleic acids are associated with the one or more therapeutic agents. The therapeutic agent may be an anti-cancer agent, or an immunostimulatory agent such as an immunostimulatory CpG nucleic acid, a nucleic acid binding moiety such as doxorubicin, and the like.

Additionally or alternatively, in some embodiments, one or more diagnostic agents are combined with the branched nucleic acids prior to crosslinking, and the resulting crosslinked branched nucleic acids are associated with the one or more diagnostic agents.

Additionally or alternatively, in some embodiments, one or more imaging agents are combined with the branched nucleic acids prior to crosslinking, and the resulting crosslinked branched nucleic acids are associated with the one or more imaging agents.

In some embodiments, the nucleic acid ligase is T4 DNA ligase. In some embodiments, the solution is aqueous solution.

In some embodiments, the lipids comprise anionic (negatively charged) lipids. In some embodiments, the lipids comprise neutral (e.g., polar or zwitterionic) lipids. In some embodiments, the lipids are homogeneous. In some embodiments, the lipids are heterogenous. In some embodiments, the lipids comprise dioleoylphosphatidylcholine (DOPC). In some embodiments, the lipids comprise dioleoylphosphatidylglycerol (DOPG).

In some embodiments, lipids comprise dioleoylphosphatidylcholine (DOPC) and dioleoylphosphatidylglycerol (DOPG). In some embodiments, the lipids comprise dioleoylphosphatidylcholine (DOPC), dioleoylphosphatidylglycerol (DOPG) and MBP.

In another aspect, the invention provides a submicron-sized particle of crosslinked nucleic acids made according to any of the foregoing methods. In some embodiments, the particle has a dimension such as an average diameter or a longest diameter ranging from about 100 nm to about 1 micron. In some embodiments, the particle has a dimension such as an average diameter or a longest diameter ranging from about 100 nm to about 500 nm. In some embodiments, the particle is dried. In some embodiments, the particle is provided in a pharmaceutically acceptable carrier, and optionally in a delivery device such as a syringe.

In another aspect, the invention provides a particle comprising crosslinked branched nucleic acids and having a dimension such as an average diameter or a longest diameter in the range of about 100 nm to about 1 micron. In some embodiment, the dimension such as the average diameter or the longest diameter is in the range of about 100 nm to about 500 nm.

In some embodiments, the particle comprises a lipid coating. In some embodiments, particle lacks a lipid coating. In some embodiments, the particle comprises one or more internal lipid layers. In some embodiments, the lipid coating or lipid layers comprise anionic (negatively charged) lipids. In some embodiments, the lipid coating or lipid layers comprise homogeneous lipids. In some embodiments, the lipid coating or lipid layers comprise heterogenous lipids.

In some embodiments, the particle comprises an agent. In some embodiments, the agent is attached to the crosslinked branched nucleic acids. In some embodiments, the agent is covalently attached to the crosslinked branched nucleic acids. In some embodiments, the agent is non-covalently attached to the crosslinked branched nucleic acids. In some embodiments, the agent is entrapped in the crosslinked branched nucleic acids.

In some embodiments, the agent is a protein having a molecular weight of about 50 kDa. In some embodiments, the particle comprises about 12 micrograms of protein per milligram of crosslinked branched nucleic acid.

In some embodiments, the particle releases an agent over a period of 20 days, 25 days, or 30 days.

In some embodiments, the particle comprises a therapeutic agent. In some embodiments, the particle comprises an anti-cancer agent. In some embodiments, the particle comprises an immunostimulatory agent. In some embodiments, the particle comprises an immunostimulatory CpG nucleic acid. In some embodiments, the particle comprises a nucleic acid binding moiety.

In some embodiments, the particle comprises doxorubicin.

In some embodiments, the particle comprises at least 50 micrograms doxorubicin per microgram of lipid. In some embodiments, the particle comprises at least 75 micrograms doxorubicin per microgram of lipid. In some embodiments, the particle comprises at least 100 micrograms doxorubicin per microgram of lipid. In some embodiments, the particle releases doxorubicin for about 1 week in the presence of serum. In some embodiments, the particle releases doxorubicin for about 2 weeks in the presence of serum. In some embodiments, the particle releases doxorubicin for about 4 weeks in the presence of serum.

In some embodiments, the particle comprises a diagnostic agent. In some embodiments, the particle comprises an imaging agent.

In some embodiments, the crosslinked branched nucleic acids comprise crosslinked branched DNA. In some embodiments, the crosslinked branched nucleic acids comprise crosslinked X-shaped nucleic acids. In some embodiments, the crosslinked branched nucleic acids comprise crosslinked Y-shaped nucleic acids. In some embodiments, the crosslinked branched nucleic acids comprise crosslinked dendrimeric nucleic acids. In some embodiments, the crosslinked branched nucleic acids are heterogeneous. In some embodiments, the crosslinked branched nucleic acids are homogeneous.

In some embodiments, the particle degrades over a period of about 20 days. In some embodiments, the particle degrades over a period of about 30 days.

In some embodiments, the particle comprises no organic solvent.

In some embodiments, the particle is dried. In some embodiments, the particle is provided in a pharmaceutically acceptable carrier, and optionally in a delivery device such as a syringe.

In another aspect, the invention provides a method comprising administering any of the foregoing nanoparticles (or submicron particles), or nanoparticles (or submicron particles) produced by any of the foregoing methods to a subject in need thereof in an effective amount.

In some embodiments, the subject has or is at risk of developing cancer. In some embodiments, the subject has or is at risk of developing an infection. In some embodiments, the subject has or is at risk of developing an allergy or asthma. In some embodiments, the subject has or is at risk of developing a neurodegenerative disorder. In some embodiments, the subject has or is at risk of developing an autoimmune disorder.

In some embodiments, the particles comprise a therapeutic agent, as described above.

In some embodiments, the particles comprise a diagnostic agent, as described above. In some embodiments, the particles comprise an imaging agent, as described above.

In some embodiments, the particles are administered systemically. In some embodiments, the particles are administered intravenously. In some embodiments, the particles are administered locally.

In some embodiments, the particles comprise an agent that is released in vivo for about 1 week. In some embodiments, the particles comprise an agent that is released in vivo for about 2 weeks. In some embodiments, the particles comprise an agent that is released in vivo for about 2 weeks. In some embodiments, the particles comprise an agent that is released in vivo for about 4 weeks.

In another aspect, the invention provides a method comprising releasing or maintaining an agent in a subject for a period of about 2-4 weeks following administration to a subject of any of the foregoing nanoparticles (or submicron particles) comprising the agent, or any nanoparticles (or submicron particles) made by any of the foregoing methods.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF DRAWINGS

It is to be understood that the Figures are not necessarily to scale, emphasis instead being placed upon generally illustrating the various concepts discussed herein.

FIG. 1 illustrates a hydrogel network formed by crosslinking of X-DNA monomers functionalized with different cargos, such as reporter dyes, functional single- or double-stranded DNA oligonucleotides, DNA-binding drugs, etc.

FIG. 2 illustrates crosslinked nucleic acid nanoparticles. Left Panel: Schematic drawing. Upper Right: Confocal microscope image of nanoparticles labeled with SYBR dye. Lower Right: Liquid cell AFM image of nanoparticle.

FIG. 3 diagrams an exemplary non-limiting synthesis process for the nanoparticles. Nanoparticles are constructed using a lipid “template”. A lipid film is mixed with branched nucleic acid monomers such as X-DNA monomers. Crosslinking agents such as T4 DNA enzyme are also included. The mixtures are put into a sonication probe (e.g., repeated with 5 to 1 watt in power) and immediately extruded under nanometer sized membrane filter (Step I). After one-day incubation, the mixture is first treated by an exonuclease and then centrifuged with 10% sucrose gradient in order to completely remove unencapsulated substrates such as free lipids and/or free nucleic acids (Step II). Optionally, the lipid coatings may be removed from the nanoparticles. To remove such lipid coatings, the nanoparticles are treated with either Triton X-100 and/or phospholipase. The resultant “naked” nanoparticles are collected using a high speed spin-down method (Step III).

FIG. 4 illustrates size evaluation (left) and confocal microscopic images (right) of nanoparticles manufactured under varying DOPC lipid amounts from 0.001 mg to 10 mg. nₗ/n_{d} is the molar ratio of lipid to DNA in the synthesis. X-DNA is fixed at approximately 1.7 mg. With increasing DOPC lipid amount, the nanoparticle size is decreased (left and right panel). The line in red color corresponds to the fitting curve about the relation between particle size and ratio of DNA and DOPC lipid.

FIG. 5 illustrates characterization of X-DNA nanogels structures. Left panel: Nucleic acids in monomers and in the resulting nanoparticles were labeled with SYBR dye (green) and lipids were labeled via rhodamine (red). Particle synthesis was carried out using X-DNAs having arms ‘capped’ with amines that prevent X-DNA crosslinking, or with X-DNAs having ‘normal’ crosslinkable arms. As shown in the confocal micrographs, both X-DNAs were entrapped in liposomes (−Triton). However, following detergent removal of the lipids (+Triton) and thus removal of the lipid coating, the non-crosslinked X-DNA disperses, while the crosslinked X-DNA remains in particle form.

FIG. 6 illustrates doxorubicin (DOX) release profile (left) and degraded nucleotide release profile (right) from nanoparticles.

FIG. 7 illustrates in vivo tumor regression mediated by doxobucin-loaded nanoparticles. 5×10⁶ Gaussia-luciferase-expressing B16F10 melanoma cells were subcutaneously injected into left flank of C57B1/6 mice. Tumors were allowed to establish and grow to ˜0.5 cm in diameter, and mice were divided randomly into groups of 8 per condition: group 1, no treatment (PBS); group 2, treated with free doxorubicin (100 μg DOX); group 3, treated with doxorubicin-imbedded liposome (100 μg DOX); and group 4, treated with doxorubicin-imbedded nucleic acid nanoparticle (100 μg DOX). Treatments were administered by intratumoral injection in 250 μl volumes. Tumors were measured every day with calipers in two dimensions. Tumor growth curves are plotted as the mean tumor area±standard deviation. Shown at bottom left is bioluminescence imaging of a mouse with an untreated tumor (left) and a mouse that received an injection of doxorubicin-imbedded nucleic acid nanoparticles (right). Animals were sacrificed after the last measurement and the tumors were excised and fixed for histology section. Slides of serial sections with no stains (right panel on bottom), showed fluorescent nanoparticles taken up the tumor (blue overlay).

FIG. 8 illustrates OVA release profiles from nanoparticles having about an 800 nm diameter. Confocal micrograph at right shows nanoparticles having about a 200 nm diameter containing 10 micrograms of fluorophore-labeled ovalbumin (pink).

### DETAILED DESCRIPTION OF INVENTION

The invention is based in part on the discovery of a method for synthesizing nano-scale particles (referred to herein generally as nanoparticles) of crosslinked nucleic acids to be used in the delivery, including sustained delivery, of a variety of agents, whether in vivo or in vitro. The particles generated by the methods of the invention are non-toxic, biodegradable and demonstrate a prolonged drug (or other active agent) release profile, making them ideal carriers for drugs (or other active agents) in vivo, including for example drugs that are otherwise toxic when delivered systemically. The particles of the invention (interchangeably referred to herein as nanoparticles, submicron particles, and nanogels) can be used to alter drug pharmacokinetics, biodistribution and bioactivity. This can facilitate the clinical use of drugs that have been heretofore too toxic for in vivo use.

The nanoparticles provided herein are a novel class of carriers made of three-dimensional crosslinked nucleic acid (e.g., DNA) networks. These nucleic acid nanoparticles may be made with a liposome-like surface coating (i.e., a lipid coat or coating, as used herein) or as uncoated (i.e., “naked”) nanoparticles. The hydrogel core of the particles is generated by crosslinking of branched nucleic acids such as double stranded ‘X’ DNA monomers, as discussed in greater detail herein.

The nanoparticles of the invention possess one or more improved characteristics as compared to existing liposome and nanoparticle technology. First, the particles may be synthesized in aqueous conditions without the use of organic solvents. This means that small molecule drugs or proteins may be retained in a native state with higher activity levels than may otherwise be possible using most existing strategies and toxic residual chemicals are minimized. Second, the crosslinked gel core of the nanoparticles can be manipulated to achieve a predictable and defined porosity based primarily on the length of the arms of the branched nucleic acids. The ability to control the porosity of the nucleic acid network allows the release rate of entrapped agents to be controlled in turn. Third, the nanoparticles may comprise free uncrosslinked arms that are coupled (or attached) to agents being delivered including drugs, imaging agents, or sensing agents. Fourth, in some instances the nucleic acids used to generate the crosslinked gel may themselves be the agent being delivered rather than simply the scaffolding that carries and retains an agent. As an example, the nucleic acids may comprise immunostimulatory oligonucleotides (e.g., CpG oligonucleotides). The Examples demonstrate several of these aspects. In addition, the Examples show loading and prolonged release of the chemotherapy drug doxorubicin and ovalbumin protein using these particles.

The invention therefore provides inter alia methods of making nucleic acid based nanoparticles, the nanoparticles themselves as well as compositions comprising such nanoparticles, and methods of using such nanoparticles.

### Nanoparticles

As used herein, nanoparticle refers to any particle having an average diameter in the range of 1 to 1000 nanometers (i.e., 1 micron). In some instances, such particles will have an average diameter in the range of 50 to 1000 nanometers, 50 to 900 nanometers, 50 to 800 nanometers, 50 to 700 nanometers, 50 to 600 nanometers, 50 to 500 nanometers, 50 to 400 nanometers, 50 to 300 nanometers, 50 to 200 nanometers, and/or 50 to 100 nanometers. The lower end of these ranges may alternatively be about 100 nanometers.

The nanoparticle may be of any shape and is not limited to a perfectly spherical shape. As an example, it may be oval or oblong. As a result, its size is referred to in terms of average diameter. As used herein, average diameter refers to the average of two or more diameter measurements. The dimensions of the microparticle may also be expressed in terms of its longest diameter or cross-section.

The nanoparticle comprises a crosslinked nucleic acid core. The crosslinked nucleic acids therefore create a three-dimensional mesh, network or gel. Accordingly, the nanoparticles are referred to herein interchangeably as nanogels. As used herein, the crosslinked nucleic acid gel may also be referred to a hydrogel since it is able to absorb water or other aqueous solution. This crosslinked nucleic acid core may act as a scaffold for retaining agent(s) and/or it may comprise agent(s) itself.

It is to be understood that the invention contemplates the use of lipid-coated as well as uncoated nanoparticles, as illustrated in the Examples. The composition of the lipid coating will depend upon the lipids used to generate the nanoparticles in the first instance. Thus, the lipid coating, if present, may comprise neutral lipids and/or anionic lipids in varying molar ratios, and such lipids may be further conjugated to other moieties such as but not limited to PEG.

The invention contemplates and the Examples demonstrate that the nanoparticles release agent for an extended period of time. The release profile may vary depending on the nature of the agent, the nature of the nanoparticles themselves including whether or not they comprise a lipid coating, the amount of agent in the nanoparticles, the size of the nanoparticles, the environment to which the nanoparticles are exposed, and the like. However, notwithstanding these various parameters, the nanoparticles are able to release agent at appreciable and medically significant levels for at least 7 days (or 1 week), at least 14 days (or 2 weeks), at least 21 days (or 3 weeks), at least 28 days (or 4 weeks), at least 35 days (or 5 weeks), or longer. In some instances, the nanoparticles are able to release agent at appreciable levels for 1-3 days. This latter release profile may be suitable for vaccination purposes. The release profile may also be defined by the rate at which the agent is being released (agent weight/time) and/or the total amount of agent released.

### Methods of Making Nanoparticles

Provided herein are methods for synthesizing the nanoparticles. Generally, the nanoparticles are produced by mixing lipids with branched nucleic acids in the presence of agents that crosslink the nucleic acids. The lipids form liposome-like particles that encapsulate the branched nucleic acids. Crosslinking agents are also encapsulated in the lipid particles and thus are able to act upon the nucleic acids. The nanoparticles typically contain agents intended for use in vivo or in vitro including without limitation therapeutic agents and diagnostic agents. These agents are typically included in the mixture of lipids and branched nucleic acids and in some instances may be combined with the branched nucleic acids prior to contact with the lipids. The relatively mild conditions used to generate nanoparticles ensure that the activity of the delivered agent will not be compromised significantly (if at all) during the process.

In one embodiment, the lipids are rehydrated in an aqueous solution with the branched nucleic acids. The method does not require the use of organic solvents and therefore the resultant nanoparticles are free of organic solvents (such as chloroform, dichloromethane, acetone and the like) that would render the nanoparticles toxic and unsuitable for in vivo use.

As discussed below in greater detail, the nanoparticles may be synthesized with a single type of branched nucleic acid or a combination of branched nucleic acids. Similarly, a single type of lipid may be used or a combination of lipids may be used. The types of branched nucleic acids, the number of sites available for crosslinking, the number of sites available for carrying payload, and the types and ratios of lipids may all be varied in accordance with the invention.

The lipids, branched nucleic acids, crosslinking agents and typically agents intended for delivery are mixed (e.g., sonicated) in order to disperse the lipids and produce liposome-like particles. Sonication times may vary but it is expected that repeated pulses lasting in duration of a few seconds, to a few minutes (depending on the volume and lipid density) will suffice. The mixture is expected to contain liposome-like particles comprising internal branched nucleic acids and crosslinking agent, empty liposome-like particles, free unencapsulated nucleic acids, and free crosslinking agent. As discussed in greater detail herein, the mole ratio of lipid to nucleic acid can impact the size of nanoparticles generated, with larger lipid to nucleic acid ratio tending to produce smaller particles. Ratios in the range of 200:1 to 5:1, or in the range of 100:1 to 5:1, or in the range of 100:1 to 10:1, or in the range of 50:1 to 10:1 can be used.

Following this step therefore the branched nucleic acids will either be encapsulated or free. As used herein, free branched nucleic acids refer to unencapsulated nucleic acids. These may exist as individual monomers or as crosslinked nucleic acids.

One step in the synthesis process requires that the entire mixture or an enriched fraction that contains the nucleic acid bearing liposome-like particles be subjected to conditions sufficient for crosslinking to occur. Such conditions and times will depend upon the type of crosslinking agent used. If the crosslinking agent is an enzyme, then the mixture can be incubated typically at neutral pH. It is expected that incubation on the order of several hours at a temperature in the range of 4-37° C. will suffice. The Examples demonstrate incubation for 24 hours at 16° C.

The synthesis process optionally includes steps to select nanoparticles of a certain size (and more likely size range). Size selection may be achieved using one or more filtration steps including for example passage through filtration membranes of decreasing pore size. Particles may be harvested from the membrane itself or from the run-through, depending on the desired size. Size selection may also be achieved using buoyant density gradient centrifugation, as well as other methods, as the invention is not limited in this regard. The particles may be selected having an average diameter in the range of 1-100 nm, 100-500 nm, 500-1000 nm, 1-1000 nm, or 100-1000 nm.

The synthesis process also typically includes steps to remove unreacted substrates and unwanted byproducts of the reaction. Unencapsulated nucleic acids may be removed by any means including chemical means (e.g., acid hydrolysis), enzymatic means (e.g., nuclease digestion such as but not limited to exonuclease digestion), and/or mechanical means (e.g., centrifugation). This may occur before or after the crosslinking step, and/or before or after size selection. Empty liposome-particles may be removed by any means including chemical means (e.g., detergent treatment such as Triton-X-100 treatment), enzymatic means (e.g., lipases such as phospholipases), and/or mechanical mans (e.g., centrifugation). These empty particles may be degraded at the same time as the lipid coating on the nucleic acid nanogels is removed, or it may occur separately. Typically lipid removal occurs following crosslinking in order to maintain the integrity of the nanogels.

The nanoparticles may be harvested at one or more steps in the synthesis process. As used herein, harvested means that the nanoparticles are collected and in some instances enriched by removal of other constituents of their environment (e.g., empty liposome-like particles or free branched nucleic acids).

The nanoparticles may be further modified or manipulated post-synthesis for example by addition of a label (e.g., for tracking or visualization). The label may be a fluorophore, or any other label that may be detected in vivo or in vitro as the particular application may require.

The method is not intended to be limited in these regards as the steps may be carried out in any manner that is convenient and suitable.

### Branched Nucleic Acids

The nanoparticles are made from branched nucleic acid complexes. As used herein, branched nucleic acid complexes are complexes of three or more nucleic acid strands in which some or all the strands hybridize to at least two other strands, as well as complexes of such complexes. Strands may comprise two regions (or sequences) each of which is complementary to regions (or sequences) of other strands. The complex may be “Y-shaped” if three strands contribute to the complex. Y-shaped nucleic acids (also referred to in the art and herein as Y nucleic acids) are described in greater detail in published US patent application US20050130180A1 to Luo et al. The complex may be “X-shaped” if four strands contribute to the complex. X-shaped nucleic acids (also referred to in the art and herein as X nucleic acids) are described in greater detail in published US patent application US20070148246A1 to Luo et al. In both instances, each strand in the complex hybridizes to two other strands. The branched nucleic acids may be dendrimeric nucleic acids, T-shaped nucleic acids, and dumbbell shaped nucleic acids, such as those illustrated and described in published US patent application US20050130180A1 to Luo et al. These references provide sequences of nucleic acids that may be used to produce branched nucleic acids such as but not limited to the Y- and X-shaped nucleic acids of the invention. In addition, these references provide sufficient guidance for how to select additional sequences to be used in the synthesis of such branched nucleic acids. Accordingly, these sequences and the rules governing the selection of these and other sequences are incorporated by reference herein in their entirety.

In some instances the generation of some of these nucleic acid forms may require one or more linear nucleic acids and some degree of ordered assembly of linear and branched nucleic acids. The art is however familiar with such processes and therefore they will not be described in any great detail herein. See for example published US patent applications US20050130180A1 and US20070148246A1, as well as Lee et al. Nat Biotech DOI:10.1038/NNANO.2009.93, 2009 (advance online publication); Um et al. Nat Materials, DOI:10.1038/nmater1741, 2006 (online publication); Um et al. Nat Protocols 1(2):995-1000, 2006.

Luo et al. have reported the production of macroscopic three-dimensional hydrogels made from crosslinking of Y-shaped DNA and X-shaped DNA. ^{1,3} The invention improves upon the report of Luo et al. at least by providing methods for generating more clinically attractive and amenable nanoparticles having crosslinked nucleic acid cores without resort to organic solvents. The submicron carriers of the invention will find broader clinical use since they can be delivered to essentially any region of the body.

The branched nucleic acids may be preformed or they may be formed from separate single-stranded nucleic acids. In the case of Y-shaped nucleic acids, typically three strands will be required each having complementarity to the other two strands. In the case of X-shaped nucleic acids, typically four strands will be required each having complementarity to at least two other strands. The length of the single-stranded oligonucleotides will vary depending on the application. In some instances, the length of the oligonucleotide strands may be 5 or more nucleotides in length, and may range from 10-100 nucleotides (or 3.4-34 nanometers, while in others it may range from 100-1000 nucleotides (or 34-340 nanometers).

The invention contemplates that individual branched nucleic acids may be comprised of DNA, PNA, LNA, combinations thereof, as well as modifications thereof. In certain instances, the branched nucleic acids do not comprise RNA or ribonucleotides. In these latter instances, the branched nucleic acids may be referred to as branched DNA in order to exclude RNA components. However it is to be understood branched DNA may comprise modifications such as modified bases, modified sugars, modified backbones, modified linkages, and generally any other modification provided such modification does not create ribonucleotide residues having RNA linkages.

Individual complexes, whether or not they include RNA, need not be “homogeneous” with respect to their nucleic acid make-up. Homogeneous complexes may however be combined with other homogeneous (but different) complexes or with heterogeneous complexes in order to form crosslinked branched nucleic acids.

It is to be understood that the longer the strands, generally the larger the “pore” or “mesh” size (or diameter) of the resulting crosslinked nucleic acid gel. This is because the branched nucleic acids crosslink with each other at their ends rather than randomly throughout their length. This ordered crosslinking allows the user to control the pore size of the resulting gels and thus to design nanoparticles suitable for particular payloads whether such payloads are small molecules or high molecular weight proteins.

As an illustration, assume an X-shaped nucleic acid having 4 arms of roughly equal length, made of strands that are each about 100 nucleotides in length. Taking in account that some nucleotides exist at the center of the X-shaped monomer and therefore do not contribute significantly to the length of the arm, each arm may have a length of about 45 nucleotides, and crosslinking two such arms together will yield dimensions of about 90 nucleotides in length. A pore may then have dimensions of 90 nucleotides by 90 nucleotides by 90 nucleotides (or about 31 nm by 31 nm by 31 nm, or about 30,000 nm³).

In some instances, pore size (diameter) may be in the range of 1-5 nm, 1-10 nm, 1-50 nm, or 1-100 nm, including about 1 nm, about 5 nm, about 10 nm, about 20 nm, about 30 nm, about 40 nm or about 50 nm.

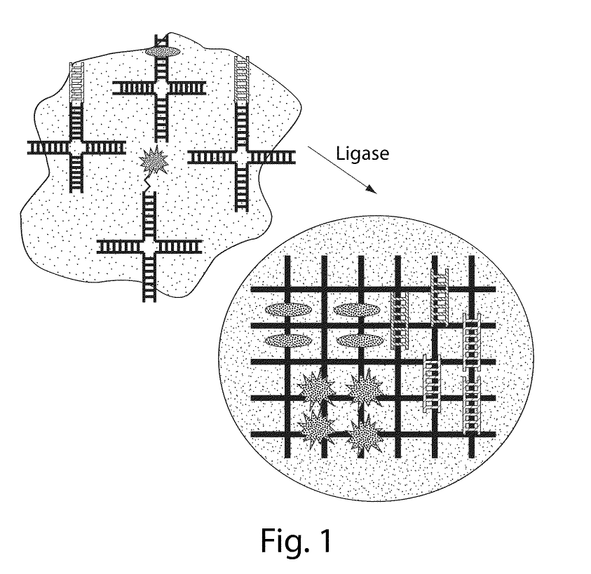

Pore size may also be controlled by the degree of crosslinking that occurs between the nucleic acid strands. As stated earlier, crosslinking occurs at the end of the arms of branched nucleic acids. X-shaped nucleic acids have 4 arms available for crosslinking, Y-shaped nucleic acids have 3 arms available for crosslinking, and dendrimeric nucleic acids have multiple arms available for crosslinking. Typically, at least some of the monomers will crosslink at 3 or more of their arms in order to form a gel or network rather than an extended linear nucleic acid polymer. Thus some monomers used to produce the crosslinking nucleic acids may have only 1 or 2 arms available for crosslinking provided that others have more than 2 arms available. If only one monomer type is used to generate nanoparticles, then it will have at least 3 arms available for crosslinking. FIG. 1 illustrates an X-shaped monomer having one “sticky end” to which a payload may be bound and three ends that are crosslinked. Mixing branched nucleic acid monomers carrying different kinds of functionalized arms facilitates the production of nanogels carrying multiple cargos or functional components.

In some instances, therefore, a mixture of X-shaped DNA monomers are used and the mixture may comprise proportions of branched nucleic acids that comprise 1, 2, 3 or 4 crosslinkable sites, with the remaining sites available for conjugation to agent or being simply inactive. As used herein, inactive sites refer to ends that are not able to be crosslinked nor conjugated to an agent, and are deliberately inactivated in order to prevent either occurrence. The number and/or frequency of these sites can impact the pore size of the resulting gel.

It will therefore be understood that the invention contemplates the use of more than one type of monomer in order to form the nanoparticles. All other things being equal, it is expected that pore size will be larger when X-shaped nucleic acid monomers having three crosslinkable ends are used as compared to X-shaped nucleic acid monomers having four crosslinkable ends.

Lipids

In order to form nanoparticles, nucleic acids are encapsulated within lipid particles. The lipids may be isolated from a naturally occurring source or they may be synthesized apart from any naturally occurring source.

The lipids may be amphipathic lipids having a hydrophilic and a hydrophobic portion. The hydrophobic portion typically orients into a hydrophobic phase, while the hydrophilic portion typically orients toward the aqueous phase. The hydrophilic portion may comprise polar or charged groups such as carbohydrates, phosphate, carboxylic, sulfato, amino, sulfhydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

Typically, the lipids are phospholipids, though other lipid membrane components such as cholesterol, sphingomyelin, cardiolipin, etc. may also be additionally or alternatively used. Phospholipids or other lipids having the ability to form spherical bilayers capable of encapsulating nucleic acids can be used in the methods provided herein. Phospholipids include without limitation phosphatidylcholine, phosphatidylethanolamine, phosphatidylglycerol, phosphatidylinositol, phosphatidylserine, and the like.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOSE). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids in order to exclude cationic lipids from the class. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palm itoyloleoyl-phosphatidylethanolamine (POPE) palm itoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some instances, modified forms of lipids may be used including forms modified with detectable labels such as fluorophores and/or reactive groups such as maleimide (e.g., dioleoyl-phosphatidylethanolamine 4-(N-maleimidomethyl)-cyclohexane-1-carboxylate (DOPE-mal) and 1,2-dipalmitoyl-sn-glycero-3-phosphoethanolamine-N-[4-(p-maleimidophenyl)butyramide] (MBP)), among others. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

The invention contemplates the use of single lipids (referred to herein as homogeneous lipids) or combinations of lipids (referred to herein as heterogeneous lipids). If combinations are used, they may be combinations of anionic lipids, combinations of neutral lipids, or combinations of anionic and neutral lipids. Such combinations may be made from a range of molar ratios. For example, neutral lipids and anionic lipids may be used in molar ratios that range from 1:100 to 100:1, or in a range from 1:10 to 10:1 or in range from 1:1 to 10:1.

In some embodiments, the lipids are combinations of zwitterionic lipids (such as DOPC) and anionic lipids (such as DOPG). In some instances, a 4:1 molar ratio of DOPC:DOPG resulted in more efficient internalization of a nanogels by melanoma cells in vitro in the absence of toxicity.

The lipids are preferably not conjugated to polyethylene glycol (PEG) prior to nanoparticle synthesis. As shown in the Examples, PEG-conjugated phospholipids appear to reduce the yield of nanoparticles in the methods described herein. However, since PEGylation is used clinically to increase the half-life of various agents including STEALTH liposomes, the instant invention contemplates modification of nanoparticles post-synthesis with PEG. This can be accomplished by using phospholipids with reactive groups (or functionalities) on their head groups (i.e., on the phosphate end) and then reacting such groups with PEG (or suitably modified PEG) post-synthesis. Reactive groups include without limitation amino groups such as primary and secondary amines, carboxyl groups, sulfhydryl groups, hydroxyl groups, aldehyde groups, azide groups, carbonyls, maleimide groups, haloacetyl (e.g., iodoacetyl) groups, imidoester groups, N-hydroxysuccinimide esters, and pyridyl disulfide groups.

The invention further contemplates using polymersome-forming block co-polymers having hydrophilic and hydrophobic blocks. Such block co-polymers can form liposome-like vesicles that entrap the branched nucleic acids and other components.

Crosslinking Agents

Crosslinking agents useful in the invention typically are able to conjugate nucleic acids to each other. In some instance, such conjugation is more specific and involves the ligation of double-stranded breaks. They include enzymes such as ligases that covalently bind nucleic acid ends to each other. In an even more specific example, crosslinking creates a phosphodiester bond between a 3′ hydroxyl of one nucleotide (and on one arm of a branched nucleic acid monomer) and a 5′ phosphate of another nucleotide (on the arm of another branched nucleic acid monomer). Exemplary enzymes include T4 DNA ligase, Thermus thermophilus ligase, Thermus acquaticus ligase, E. coli ligase, and Pyrococcus ligase. These and other enzymes may be used alone or in combination. Ligation carried out by enzymes is typically carried out between 4-37° C. Since the nanoparticles are intended for in vivo use in some instances, it is important that the crosslinking agents (and any other entities) present in or on the nanoparticles be non-toxic.

The invention further contemplates the use of nucleic acids including branched nucleic acids that are functionalized at their ends in order to effect crosslinking. For example, the nucleic acids may be used that comprise complementary chemical reactive groups (such as acrylate and amine) that would crosslink to each other through for example Michael addition, disulfide formation between thiolated nucleic acids, or other water-compatible crosslinking reactions, of which a variety are known in the art.

Nucleic Acids

The nucleic acid complexes may comprise naturally occurring and/or non-naturally occurring nucleic acids. If naturally occurring, the nucleic acids may be isolated from natural sources or they may be synthesized apart from their naturally occurring sources. Non-naturally occurring nucleic acids are synthetic.

The terms “nucleic acid”, “oligonucleotide”, and “oligodeoxyribonucleotide” are used interchangeably to mean multiple nucleotides (i.e., molecules comprising a sugar (e.g. a deoxyribose) linked to a phosphate group and to an exchangeable organic base, which is either a pyrimidine (e.g., cytosine (C), thymidine (T) or uracil (U)) or a purine (e.g., adenine (A) or guanine (G)). In some instances, the nucleic acid is not RNA or an oligoribonucleotide. Thus, in some instances, the nucleic acid complex does not comprise RNA or oligoribonucleotides. In these instances, the branched nucleic acids may be referred to as branched DNA or branched DNA complexes. DNA complexes however may still comprise base, sugar and backbone modifications.

Modifications

The complexes may be made of DNA, modified DNA, and combinations thereof. The oligodeoxyribonucleotides (also referred to herein as oligonucleotides) that are comprised by the complex may have a homogeneous or heterogeneous (i.e., chimeric) backbone. The backbone may be a naturally occurring backbone such as a phosphodiester backbone or it may comprise backbone modification(s). In some instances, backbone modification results in a longer half-life for the oligonucleotides due to reduced nuclease-mediated degradation. This is turn results in a longer half-life and extended release profiles of the crosslinked complexes. Examples of suitable backbone modifications include but are not limited to phosphorothioate modifications, phosphorodithioate modifications, p-ethoxy modifications, methylphosphonate modifications, methylphosphorothioate modifications, alkyl- and aryl-phosphates (in which the charged phosphonate oxygen is replaced by an alkyl or aryl group), alkylphosphotriesters (in which the charged oxygen moiety is alkylated), peptide nucleic acid (PNA) backbone modifications, locked nucleic acid (LNA) backbone modifications, and the like. These modifications may be used in combination with each other and/or in combination with phosphodiester backbone linkages.

Alternatively or additionally, the oligonucleotides may comprise other modifications including modifications at the base or the sugar moieties. Examples include nucleic acids having sugars which are covalently attached to low molecular weight organic groups other than a hydroxyl group at the 3′ position and other than a phosphate group at the 5′ position (e.g., a 2′-O-alkylated ribose), nucleic acids having sugars such as arabinose instead of ribose. Nucleic acids also embrace substituted purines and pyrimidines such as C-5 propyne modified bases (Wagner et al., Nature Biotechnology 14:840-844, 1996). Other purines and pyrimidines include but are not limited to 5-methylcytosine, 2-aminopurine, 2-amino-6-chloropurine, 2,6-diaminopurine, hypoxanthine. Other such modifications are well known to those of skill in the art.

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863, and alkylphosphotriesters (in which the charged oxygen moiety is alkylated as described in U.S. Pat. No. 5,023,243 and European Patent No. 092574) can be prepared by automated solid phase synthesis using commercially available reagents. Methods for making other DNA backbone modifications and substitutions have been described (Uhlmann, E. and Peyman, A., Chem. Rev. 90:544, 1990; Goodchild, J., Bioconjugate Chem. 1:165, 1990).

Nucleic acids can be synthesized de novo using any of a number of procedures known in the art including for example the b-cyanoethyl phosphoramidite method (Beaucage and Caruthers Tet. Let. 22:1859, 1981), and the nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated nucleic acid synthesizers available in the market. These nucleic acids are referred to as synthetic nucleic acids.

Alternatively, oligonucleotides may be generated from larger nucleic acids such as but not limited to plasmids. Nucleic acids can be prepared from existing nucleic acid sequences (e.g., genomic or cDNA) using known techniques, such as those employing restriction enzymes, exonucleases or endonucleases. Nucleic acids prepared in this manner are referred to as isolated nucleic acid. An isolated nucleic acid generally refers to a nucleic acid which is separated from components which it is normally associated with in nature. As an example, an isolated nucleic acid may be one which is separated from a cell, from a nucleus, from mitochondria, or from chromatin.

Agents

The nanoparticles will typically contain agents that are intended for use in vivo and/or in vitro. As used herein, an agent is any atom, molecule or compound that can be used to provide benefit to a subject (including without limitation prophylactic or therapeutic benefit) or that can be used for diagnosis and/or detection (for example, imaging) in vivo, or that may be used for effect in an in vitro setting (for example, a tissue or organ culture, a clean up process, and the like). The agents may be without limitation therapeutic agents and diagnostic agents. Non-exhaustive lists are provided below.

The agents may be covalently or non-covalently attached to the crosslinked nucleic acids. If covalently or non-covalently attached, in some instances, the agents may be combined with the branched nucleic acids prior to contact with the lipids. Covalent attachment of agents to branched nucleic acids may involve the use of bonds that can be cleaved under physiological conditions or that can be caused to cleave specifically upon application of a stimulus such as light, whereby the agent can be released. Readily cleavable bonds include readily hydrolyzable bonds, for example, ester bonds, amide bonds and Schiff's base-type bonds. Bonds which are cleavable by light are known. In certain instances, the agent may be inactive in its bound form and activated only when released.

Non-covalently attached agents include those having affinity for nucleic acids (and thus having nucleic acid binding activity). Examples of such agents include without limitation certain drugs including certain cancer chemotherapies that act by binding to and damaging DNA, certain proteins (such as DNA repair enzymes, DNA polymerases, restriction endonucleases, topoisomerases, telomerases, and the like), nucleic acids or nucleic acid derivatives (e.g., PNA) that bind to other nucleic acids via Watson-Crick binding and/or Hoogsteen binding, non-nucleic acid probes that bind in the major and/or minor groove of the nucleic acid, and the like. The Examples illustrate the encapsulation of doxorubicin, an anti-cancer agent that binds DNA. Alternatively, the agents may be physically entrapped in the crosslinked nucleic acids, typically as a result of their size relative to the “pore” or “mesh” size of the resulting crosslinked nucleic acids.

As demonstrated in the Examples, the nanoparticles of the invention possess long-term release profiles for small molecule agents with affinity for DNA such as doxorubicin as well as higher molecular weight proteins such as ovalbumin. The mechanism by which agents are released from the nanoparticle will depend in part on the mechanism by which the agent is retained in the nanoparticle in the first instance.

In one instance, the agent may be entrapped within the gel in the absence of covalent or non-covalent bonds. In this situation, degradation of the gel (and nucleic acids) in whole or in part must occur in order to release the agent. Degradation of the gel resulting in greater pore size can be another route through which the agents are released. This may be the case for example with high molecular weight agents such as proteins.

In another instance, the agent may be non-covalently attached to the crosslinked nucleic acids, and release from the nanoparticles may occur as the agent dissociates from the nucleic acids or functional or reactive groups on the nucleic acids. Since the nanoparticles are likely to be hydrated, the agent may simply diffuse away from its reactive site, into the aqueous solution, and out of the nanoparticle. If the agent is retained in the nanoparticle by virtue of its ability to bind to nucleic acids (e.g., it is a nucleic acid binding agent), a similar process is envisioned whereby the agent will dissociate from the nucleic acid and then diffuse out of the nanoparticle whether or not the nucleic acid gel has degraded. In an alternative manner, the nucleic acid gel may degrade, leaving the nucleic acid binding agent without a binding partner and able to diffuse out of the nanoparticle.

If the agent is covalently bound to the nucleic acid gel, then its release may come about by degradation of the gel. Alternatively, if the covalent bond is cleavable in response to physiological stimuli, then the agent may be released through cleavage of such bond. In either situation, it is possible that the agent may retain a part of the nucleic acid gel or the bond constituents but it is not expected that either will negatively impact the activity of the agent or be toxic to the subject.

The invention contemplates in some aspects the delivery of agents either systemically or to localized regions, tissues or cells. Any agent may be delivered using the methods of the invention provided that it can be loaded into the nanoparticles provided herein and can withstand the synthesis processes described herein. Since such processes are relatively innocuous, it is expected that virtually any agent may be used provided it can be encapsulated in the nanoparticles provided herein.

The nanoparticles may be synthesized and stored in, for example, a lyophilized and optionally frozen form. The agents should be stable during such storage procedures and times.

The agents may be naturally occurring or non-naturally occurring. Naturally occurring agents include those capable of being synthesized by the subjects to whom the nanoparticles are administered. Non-naturally occurring are those that do not exist in nature normally, whether produced by plant, animal, microbe or other living organism.

The agent may be without limitation a chemical compound including a small molecule, a protein, a polypeptide, a peptide, a nucleic acid, a virus-like particle, a steroid, a proteoglycan, a lipid, a carbohydrate, and analogs, derivatives, mixtures, fusions, combinations or conjugates thereof. The agent may be a prodrug that is metabolized and thus converted in vivo to its active (and/or stable) form. The invention further contemplates the loading of more than one type of agent in a nanoparticle and/or the combined use of nanoparticles comprising different agents.

One class of agents is peptide-based agents such as (single or multi-chain) proteins and peptides. Examples include antibodies, single chain antibodies, antibody fragments, enzymes, co-factors, receptors, ligands, transcription factors and other regulatory factors, some antigens (as discussed below), cytokines, chemokines, hormones, and the like.

Another class of agents that can be delivered using the nanoparticles of the invention includes chemical compounds that are non-naturally occurring.

A variety of agents that are currently used for therapeutic or diagnostic purposes can be delivered according to the invention and these include without limitation imaging agents, immunomodulatory agents such as immunostimulatory agents and immunoinhibitory agents (e.g., cyclosporine), antigens, adjuvants, cytokines, chemokines, anti-cancer agents, anti-infective agents, nucleic acids, antibodies or fragments thereof, fusion proteins such as cytokine-antibody fusion proteins, Fc-fusion proteins, analgesics, opioids, enzyme inhibitors, neurotoxins, hypnotics, anti-histamines, lubricants, tranquilizers, anti-convulsants, muscle relaxants, anti-Parkinson agents, anti-spasmodics, muscle contractants including channel blockers, miotics and anti-cholinergics, anti-glaucoma compounds, modulators of cell-extracellular matrix interactions including cell growth inhibitors and anti-adhesion molecules, vasodilating agents, inhibitors of DNA, RNA or protein synthesis, anti-hypertensives, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, targeting agents, neurotransmitters, proteins, cell response modifiers, and vaccines.

Imaging Agents. As used herein, an imaging agent is an agent that emits signal directly or indirectly thereby allowing its detection in vivo. Imaging agents such as contrast agents and radioactive agents that can be detected using medical imaging techniques such as nuclear medicine scans and magnetic resonance imaging (MRI). Imaging agents for magnetic resonance imaging (MRI) include Gd(DOTA), iron oxide or gold nanoparticles; imaging agents for nuclear medicine include ²⁰¹Tl, gamma-emitting radionuclide 99 mTc; imaging agents for positron-emission tomography (PET) include positron-emitting isotopes, (18)F-fluorodeoxyglucose ((18)FDG), (18)F-fluoride, copper-64, gadoamide, and radioisotopes of Pb(II) such as 203Pb, and 11In; imaging agents for in vivo fluorescence imaging such as fluorescent dyes or dye-conjugated nanoparticles. In other embodiments, the agent to be delivered is conjugated, or fused to, or mixed or combined with an imaging agent.

Immunostimulatory Agents. As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod, imidazoquinoline, resiquimod, nucleic acids comprising an unmethylated CpG dinucleotide, monophosphoryl lipid A or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

Antigens. The antigen may be without limitation a cancer antigen, a self antigen, a microbial antigen, an allergen, or an environmental antigen. The antigen may be peptide, lipid, or carbohydrate in nature, but it is not so limited.

Cancer Antigens. A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T-cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCASI, α-fetoprotein, E-cadherin, α-catenin, β-catenin, γ-catenin, p120ctn, gp100^{Pmell17}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

Microbial Antigens. Microbial antigens are antigens derived from microbial species such as without limitation bacterial, viral, fungal, parasitic and mycobacterial species. As such, microbial antigens include bacterial antigens, viral antigens, fungal antigens, parasitic antigens, and mycobacterial antigens. Examples of bacterial, viral, fungal, parasitic and mycobacterial species are provided herein. The microbial antigen may be part of a microbial species or it may be the entire microbe.

Allergens. An allergen is an agent that can induce an allergic or asthmatic response in a subject. Allergens include without limitation pollens, insect venoms, animal dander dust, fungal spores and drugs (e.g. penicillin). Examples of natural, animal and plant allergens include but are not limited to proteins specific to the following genera: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Adjuvants. The adjuvant may be without limitation saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic)

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod, resiquimod). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages. In these latter instances, the adjuvant may be incorporated or be an integral part of the nucleic acid gel and will be released as the gel is degraded.

Immunoinhibitory Agents. As used herein, an immunoinhibitory agent is an agent that inhibits an immune response in a subject to whom it is administered, whether alone or in combination with another agent. Examples include steroids, retinoic acid, dexamethasone, cyclophosphamide, anti-CD3 antibody or antibody fragment, and other immunosuppressants.

Growth Factors. The nanoparticles may comprise growth factors including without limitation VEGF-A, VEGF-C P1GF, KDR, EGF, HGF, FGF, angiopoietin-1, cytokines, endothelial nitric oxide synthases eNOS and iNOS, G-CSF, GM-CSF, VEGF, aFGF, SCF (c-kit ligand), bFGF, TNF, heme oxygenase, AKT (serine-threonine kinase), HIF.alpha.(hypoxia inducible factor), Del-1 (developmental embryonic locus-1), NOS (nitric oxide synthase), BMP's (bone morphogenic proteins), SERCA2a (sarcoplasmic reticulum calcium ATPase), beta-2-adrenergic receptor, SDF-1, MCP-1, other chemokines, interleukins and combinations thereof.

Anti-Cancer Agents. As used herein, an anti-cancer agent is an agent that at least partially inhibits the development or progression of a cancer, including inhibiting in whole or in part symptoms associated with the cancer even if only for the short term. Several anti-cancer agents can be categorized as DNA damaging agents and these include topoisomerase inhibitors (e.g., etoposide, ramptothecin, topotecan, teniposide, mitoxantrone), DNA alkylating agents (e.g., cisplatin, mechlorethamine, cyclophosphamide, ifosfamide, melphalan, chorambucil, busulfan, thiotepa, carmustine, lomustine, carboplatin, dacarbazine, procarbazine), DNA strand break inducing agents (e.g., bleomycin, doxorubicin, daunorubicin, idarubicin, mitomycin C), anti-microtubule agents (e.g., vincristine, vinblastine), anti-metabolic agents (e.g., cytarabine, methotrexate, hydroxyurea, 5-fluorouracil, floxuridine, 6-thioguanine, 6-mercaptopurine, fludarabine, pentostatin, chlorodeoxyadenosine), anthracyclines, vinca alkaloids. or epipodophyllotoxins.

Examples of anti-cancer agents include without limitation Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Bortezomib (VELCADE); Brequinar Sodium; Bropirimine; Busulfan; Cactinomycin; Calusterone; Caracemide; Carbetimer; Carboplatin (a platinum-containing regimen); Carmustine; Carubicin Hydrochloride; Carzelesin; Cedefingol; Chlorambucil; Cirolemycin; Cisplatin (a platinum-containing regimen); Cladribine; Crisnatol Mesylate; Cyclophosphamide; Cytarabine; Dacarbazine; Dactinomycin; Daunorubicin; Decitabine; Dexormaplatin; Dezaguanine; Diaziquone; Docetaxel (TAXOTERE); Doxorubicin (DOXIL); Droloxifene; Dromostanolone; Duazomycin; Edatrexate; Eflornithine; Elsamitrucin; Enloplatin; Enpromate; Epipropidine; Epirubicin; Erbulozole; Erlotinib (TARCEVA), Esorubicin; Estramustine; Etanidazole; Etoposide; Etoprine; Fadrozole; Fazarabine; Fenretinide; Floxuridine; Fludarabine; 5-Fluorouracil; Flurocitabine; Fosquidone; Fostriecin; Gefitinib (IRESSA), Gemcitabine; Hydroxyurea; Idarubicin; Ifosfamide; Ilmofosine; Imatinib mesylate (GLEEVAC); Interferon alpha-2a; Interferon alpha-2b; Interferon alpha-n1; Interferon alpha-n3; Interferon beta-I a; Interferon gamma-I b; Iproplatin; Irinotecan; Lanreotide; Lenalidomide (REVLIMID, REVIMID); Letrozole; Leuprolide; Liarozole; Lometrexol; Lomustine; Losoxantrone; Masoprocol; Maytansine; Mechlorethamine; Megestrol; Melengestrol; Melphalan; Menogaril; Mercaptopurine; Methotrexate; Metoprine; Meturedepa; Mitindomide; Mitocarcin; Mitocromin; Mitogillin; Mitomalcin; Mitomycin; Mitosper; Mitotane; Mitoxantrone; Mycophenolic Acid; Nocodazole; Nogalamycin; Ormaplatin; Oxisuran; Paclitaxel; Pemetrexed (ALIMTA), Pegaspargase; Peliomycin; Pentamustine; Pentomone; Peplomycin; Perfosfamide; Pipobroman; Piposulfan; Piritrexim Isethionate; Piroxantrone; Plicamycin; Plomestane; Porfimer; Porfiromycin; Prednimustine; Procarbazine; Puromycin; Pyrazofurin; Riboprine; Rogletimide; Safingol; Semustine; Simtrazene; Sitogluside; Sparfosate; Sparsomycin; Spirogermanium; Spiromustine; Spiroplatin; Streptonigrin; Streptozocin; Sulofenur; Talisomycin; Tamsulosin; Taxol; Taxotere; Tecogalan; Tegafur; Teloxantrone; Temoporfin; Temozolomide (TEMODAR); Teniposide; Teroxirone; Testolactone; Thalidomide (THALOMID) and derivatives thereof; Thiamiprine; Thioguanine; Thiotepa; Tiazofurin; Tirapazamine; Topotecan; Toremifene; Trestolone; Triciribine; Trimetrexate; Triptorelin; Tubulozole; Uracil Mustard; Uredepa; Vapreotide; Verteporfin; Vinblastine; Vincristine; Vindesine; Vinepidine; Vinglycinate; Vinleurosine; Vinorelbine; Vinrosidine; Vinzolidine; Vorozole; Zeniplatin; Zinostatin; Zorubicin.

The anti-cancer agent may be an enzyme inhibitor including without limitation tyrosine kinase inhibitor, a CDK inhibitor, a MAP kinase inhibitor, or an EGFR inhibitor. The tyrosine kinase inhibitor may be without limitation Genistein (4′,5,7 trihydroxyisoflavone), Tyrphostin 25 (3,4,5-trihydroxyphenyl), methylene]-propanedinitrile, Herbimycin A, Daidzein (4′,7-dihydroxyisoflavone), AG-126, trans-1-(3′-carboxy-4′-hydroxyphenyl)-2-(2″,5″-dihydroxy-phenyl)ethane, or HDBA (2-Hydroxy5-(2,5-Dihydroxybenzylamino)-2-hydroxybenzoic acid. The CDK inhibitor may be without limitation p21, p27, p57, p15, p16, p18, or p19. The MAP kinase inhibitor may be without limitation KY12420 (C₂₃H₂₄O₈), CNI-1493, PD98059, or 4-(4-Fluorophenyl)-2-(4-methylsulfinyl phenyl)-5-(4-pyridyl) 1H-imidazole. The EGFR inhibitor may be without limitation erlotinib (TARCEVA), gefitinib (IRESSA), WHI-P97 (quinazoline derivative), LFM-A12 (leflunomide metabolite analog), ABX-EGF, lapatinib, canertinib, ZD-6474 (ZACTIMA), AEE788, and AG1458.

The anti-cancer agent may be a VEGF inhibitor including without limitation bevacizumab (AVASTIN), ranibizumab (LUCENTIS), pegaptanib (MACUGEN), sorafenib, sunitinib (SUTENT), vatalanib, ZD-6474 (ZACTIMA), anecortave (RETAANE), squalamine lactate, and semaphorin.

The anti-cancer agent may be an antibody or an antibody fragment including without limitation an antibody or an antibody fragment including but not limited to bevacizumab (AVASTIN), trastuzumab (HERCEPTIN), alemtuzumab (CAMPATH, indicated for B cell chronic lymphocytic leukemia,), gemtuzumab (MYLOTARG, hP67.6, anti-CD33, indicated for leukemia such as acute myeloid leukemia), rituximab (RITUXAN), tositumomab (BEXXAR, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX, indicated for ovarian cancer), edrecolomab (PANOREX), daclizumab (ZENAPAX), palivizumab (SYNAGIS, indicated for respiratory conditions such as RSV infection), ibritumomab tiuxetan (ZEVALIN, indicated for Non-Hodgkin's lymphoma), cetuximab (ERBITUX), MDX-447, MDX-22, MDX-220 (anti-TAG-72), IOR-05, IOR-T6 (anti-CD1), IOR EGF/R3, celogovab (ONCOSCINT OV 103), epratuzumab (LYMPHOCIDE), pemtumomab (THERAGYN), and Gliomab-H (indicated for brain cancer, melanoma).

Anti-Infective Agents. The agent may be an anti-infective agent including without limitation an anti-bacterial agent, an anti-viral agent, an anti-parasitic agent, an anti-fungal agent, and an anti-mycobacterial agent.

Anti-bacterial agents may be without limitation β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, or quinolines.

Other anti-bacterials may be without limitation Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefmenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; or Zorbamycin.

Anti-mycobacterial agents may be without limitation Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate or Trecator-SC (Ethionamide).

Anti-viral agents may be without limitation amantidine and rimantadine, ribivarin, acyclovir, vidarabine, trifluorothymidine, ganciclovir, zidovudine, retinovir, and interferons.

Anti-viral agents may be without limitation further include Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime or integrase inhibitors.

Anti-fungal agents may be without limitation imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhbitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); or sulfated naphthylamine (e.g., suramin).

Other anti-infective agents may be without limitation Difloxacin Hydrochloride; Lauryl Isoquinolinium Bromide; Moxalactam Disodium; Ornidazole; Pentisomicin; Sarafloxacin Hydrochloride; Protease inhibitors of HIV and other retroviruses; Integrase Inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride: Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; or Troclosene Potassium.

Nucleic Acid Agents. Nucleic acids that can be delivered to a subject according to the invention include naturally or non-naturally occurring DNA (including cDNA, genomic DNA, nuclear DNA, mitochondrial DNA), RNA, oligonucleotides, a triple-helix forming molecule, immunostimulatory nucleic acids such as those described in U.S. Pat. No. 6,194,388 (the teachings of which relating to immunostimulatory CpG nucleic acids are incorporated herein by reference), miRNA, siRNA and antisense oligonucleotides used to modulate gene expression, aptamers, ribozymes, a gene or gene fragment, a regulatory sequence, including analogs, derivatives, and combinations thereof. These nucleic acids may be administered neat or complexed to another entity, for example in order to facilitate their binding to and/or uptake by target tissues and/or cells.

Other Agents. The agent may be without limitation adrenergic agent; adrenocortical steroid; adrenocortical suppressant; alcohol deterrent; aldosterone antagonist; ammonia detoxicant; amino acid; amylotropic lateral sclerosis agent; anabolic; analeptic; analgesic; androgen; anesthetic; anorectic; anorexic; anterior pituitary activator; anterior pituitary suppressant; anthelmintic; anti-acne agent; anti-adrenergic; anti-allergic; anti-amebic; anti-androgen; anti-anemic; anti-anginal; anti-anxiety; anti-arthritic; anti-asthmatic including β-adrenergic agonists, methylxanthines, mast cell stabilizing agents, anticholinergics, adrenocortical steroids such as glucocorticoids; anti-atherosclerotic; anticholelithic; anticholelithogenic; anticholinergic; anticoagulant; anticoccidal; anticonvulsant; antidepressant; antidiabetic; antidiarrheal; antidiuretic; antidote; antidyskinetic; anti-emetic; anti-epileptic; anti-estrogen; antifibrinolytic; antiglaucoma; antihemorrhagic; antihemorrheologic; antihistamine; antihyperlipidemic; antihyperlipoproteinemic; antihypertensive; antihypotensive; anti-infective; anti-inflammatory; antikeratinizing agent; antimigraine; antimitotic; antimycotic; antinauseant; antineutropenic; antiobsessional agent; antioxidant; antiparkinsonian; antiperistaltic; antipneumocystic; antiprostatic hypertrophy agent; antiprotozoal; antipruritic; antipsoriatic; antipsychotic; antirheumatic; antischistosomal; antiseborrheic; antisecretory; antispasmodic; antithrombotic; antitussive; anti-ulcerative; anti-urolithic; appetite suppressant; blood glucose regulator; bone resorption inhibitor; bronchodilator; carbonic anhydrase inhibitor; cardiac depressant; cardioprotectant; cardiotonic; cardiovascular agent; cerebral ischemia agent; choleretic; cholinergic; cholinergic agonist; cholinesterase deactivator; coccidiostat; cognition adjuvant; cognition enhancer; conjunctivitis agent; contrast agent; depressant; diagnostic aid; diuretic; dopaminergic agent; ectoparasiticide; emetic; enzyme inhibitor; estrogen; estrogen receptor agonist; fibrinolytic; fluorescent agent; free oxygen radical scavenger; gastric acid suppressant; gastrointestinal motility effector; geriatric agent; glucocorticoid; gonad-stimulating principle; hair growth stimulant; hemostatic; herbal active agent; histamine H2 receptor antagonists; hormone; hypocholesterolemic; hypoglycemic; hypolipidemic; hypotensive; HMGCoA reductase inhibitor; impotence therapy adjunct; inflammatory bowel disease agent; keratolytic; LHRH agonist; liver disorder agent; luteolysin; memory adjuvant; mental performance enhancer; mineral; mood regulator; mucolytic; mucosal protective agent; multiple sclerosis agent; mydriatic; nasal decongestant; neuroleptic; neuromuscular blocking agent; neuroprotective; NMDA antagonist; non-hormonal sterol derivative; nutrient; oxytocic; Paget's disease agent; plasminogen activator; platelet activating factor antagonist; platelet aggregation inhibitor; post-stroke and post-head trauma agents; progestin; prostaglandin; prostate growth inhibitor; prothyrotropin; psychotropic; radioactive agent; relaxant; rhinitis agent; scabicide; sclerosing agent; sedative; sedative-hypnotic; selective adenosine Al antagonist; sequestering agents; serotonin antagonist; serotonin inhibitor; serotonin receptor antagonist; steroid; stimulant; suppressant; thyroid hormone; thyroid inhibitor; thyromimetic; tranquilizer; unstable angina agent; uricosuric; vasoconstrictor; vasodilator; vulnerary; wound healing agent; or xanthine oxidase inhibitor.

### In Vitro Use

The invention further contemplates in vitro applications such as cell culturing and tissue engineering, that require or for which it would be more convenient to have a constant source of one or more agents such as but not limited to cell growth factors, and the like.

### Subjects

When the nanoparticles are used in vivo, the invention can be practiced in virtually any subject type that is likely to benefit prophylactically, therapeutically, or prognostically from the delivery of agents using the nanoparticles of the invention as contemplated herein.

Human subjects are preferred subjects in some embodiments of the invention. Subjects also include animals such as household pets (e.g., dogs, cats, rabbits, ferrets, etc.), livestock or farm animals (e.g., cows, pigs, sheep, chickens and other poultry), horses such as thoroughbred horses, laboratory animals (e.g., mice, rats, rabbits, etc.), and the like. Subjects also include fish and other aquatic species.

The subjects to whom the agents are delivered may be normal subjects. Alternatively they may have or may be at risk of developing a condition that can be diagnosed or that can benefit or that can be prevented from systemic or localized delivery of one or more particular agents. Such conditions include cancer (e.g., solid tumor cancers), infections (particularly infections localized to particular regions or tissues in the body), autoimmune disorders, allergies or allergic conditions, asthma, transplant rejection, diabetes, heart disease, and the like.

In some instances, the agents are delivered to prevent the onset of a condition whether or not such condition is considered a disorder. For example, the agents may be contraceptives which when embedded in the nanoparticles of the invention are released for a prolonged period of time. This obviates the need to take contraceptives on a daily or weekly time period. In a similar manner, the nanoparticles described herein may be used in subject that are prone to memory loss (e.g., the elderly) resulting in missed medication. By delivering the medication in nanoparticle(s) form that provides an extended release profile of the agent(s), then the subject is more likely to receive the medication at the dosages at which it was prescribed.

Tests for diagnosing various of the conditions embraced by the invention are known in the art and will be familiar to the ordinary medical practitioner. These laboratory tests include without limitation microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner will generally also take a full history and conduct a complete physical examination in addition to running the laboratory tests listed above.

A subject having a cancer is a subject that has detectable cancer cells. A subject at risk of developing a cancer is a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

Subjects having an infection are those that exhibit symptoms thereof including without limitation fever, chills, myalgia, photophobia, pharyngitis, acute lymphadenopathy, splenomegaly, gastrointestinal upset, leukocytosis or leukopenia, and/or those in whom infectious pathogens or byproducts thereof can be detected.

A subject at risk of developing an infection is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

The subject may have or may be at risk of developing an infection such as a bacterial infection, a viral infection, a fungal infection, a parasitic infection or a mycobacterial infection. In these embodiments, the nanoparticles may comprise an anti-microbial agent such as an anti-bacterial agent, an anti-viral agent, an anti-fungal agent, an anti-parasitic agent, or an anti-mycobacterial agent and the cell carriers (e.g., the T cells) may be genetically engineered to produce another agent useful in stimulating an immune response against the infection, or potentially treating the infection.

### Cancer

The invention contemplates administration of the nanoparticles of the invention to subjects having or at risk of developing a cancer including for example a solid tumor cancer. The cancer may be carcinoma, sarcoma or melanoma. Carcinomas include without limitation to basal cell carcinoma, biliary tract cancer, bladder cancer, breast cancer, cervical cancer, choriocarcinoma, CNS cancer, colon and rectum cancer, kidney or renal cell cancer, larynx cancer, liver cancer, small cell lung cancer, non-small cell lung cancer (NSCLC, including adenocarcinoma, giant (or oat) cell carcinoma, and squamous cell carcinoma), oral cavity cancer, ovarian cancer, pancreatic cancer, prostate cancer, skin cancer (including basal cell cancer and squamous cell cancer), stomach cancer, testicular cancer, thyroid cancer, uterine cancer, rectal cancer, cancer of the respiratory system, and cancer of the urinary system.

Sarcomas are rare mesenchymal neoplasms that arise in bone (osteosarcomas) and soft tissues (fibrosarcomas). Sarcomas include without limitation liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., not bone) Ewing's sarcoma, and primitive neuroectodermal tumor), synovial sarcoma, angiosarcomas, hemangiosarcomas, lymphangiosarcomas, Kaposi's sarcoma, hemangioendothelioma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), and chondrosarcoma.

Melanomas are tumors arising from the melanocytic system of the skin and other organs. Examples of melanoma include without limitation lentigo maligna melanoma, superficial spreading melanoma, nodular melanoma, and acral lentiginous melanoma.

The cancer may be a solid tumor lymphoma. Examples include Hodgkin's lymphoma, Non-Hodgkin's lymphoma, and B cell lymphoma.

The cancer may be without limitation bone cancer, brain cancer, breast cancer, colorectal cancer, connective tissue cancer, cancer of the digestive system, endometrial cancer, esophageal cancer, eye cancer, cancer of the head and neck, gastric cancer, intra-epithelial neoplasm, melanoma neuroblastoma, Non-Hodgkin's lymphoma, non-small cell lung cancer, prostate cancer, retinoblastoma, or rhabdomyosarcoma.

### Infection

The invention contemplates administration of the nanoparticles of the invention to subjects having or at risk of developing an infection such as a bacterial infection, a viral infection, a fungal infection, a parasitic infection or a mycobacterial infection.

The bacterial infection may be without limitation an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteriodes infection, H. pylori infection, or anthrax infection.

The mycobacterial infection may be without limitation tuberculosis or leprosy respectively caused by the M. tuberculosis and M. leprae species.

The viral infection may be without limitation a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection, SARS infection or avian flu infection.

The fungal infection may be without limitation candidiasis, ringworm, histoplasmosis, blastomycosis, paracoccidioidomycosis, crytococcosis, aspergillosis, chromomycosis, mycetoma infections, pseudallescheriasis, or tinea versicolor infection.

The parasite infection may be without limitation amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, or Trichuris trichuria infection.

### Allergy and Asthma

The invention contemplates administration of the nanoparticles of the invention to subjects having or at risk of developing an allergy or asthma. An allergy is an acquired hypersensitivity to an allergen. Allergic conditions include but are not limited to eczema, allergic rhinitis or coryza, hay fever, bronchial asthma, urticaria (hives) and food allergies, and other atopic conditions. Allergies are generally caused by IgE antibody generation against harmless allergens. Asthma is a disorder of the respiratory system characterized by inflammation, narrowing of the airways and increased reactivity of the airways to inhaled agents. Asthma is frequently, although not exclusively, associated with atopic or allergic symptoms. Administration of Th1 cytokines, such as IL-12 and IFN-gamma, according to the invention can be used to treat allergy or asthma.

### Autoimmune Disease

The invention contemplates administration of the nanoparticles of the invention to subjects having or at risk of developing an autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases are generally considered to be Th1 biased. As a result, induction of a Th2 immune response or Th2 like cytokines can be beneficial. Such cytokines include IL-4, IL-5 and IL-10.

Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjogren's syndrome, insulin resistance, and autoimmune diabetes mellitus.

### Transplant Therapy

The methods provided herein may also be used to modulate immune responses following transplant therapy. Transplant success is often limited by rejection of the transplanted tissue by the body's immune system. As a result, transplant recipients are usually immunosuppressed for extended periods of time in order to allow the transplanted tissue to survive. The invention contemplates localized (e.g., to transplant sites, organs or tissues) or in some instances systemic delivery of immunomodulators, and particularly immunoinhibitory agents, in order to minimize transplant rejection. Thus, the invention contemplates administration of the nanoparticles to subjects that are going to undergo, are undergoing, or have undergone a transplant.

The foregoing lists are not intended to be exhaustive but rather exemplary. Those of ordinary skill in the art will identify other examples of each condition type that are amenable to prevention and treatment using the methods of the invention.

### Effective Amounts, Regimens, Formulations

The agents are administered in effective amounts. An effective amount is a dosage of the agent sufficient to provide a medically desirable result. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment.

For example, if the subject has a tumor, an effective amount may be that amount that reduces the tumor volume or load (as for example determined by imaging the tumor). Effective amounts may also be assessed by the presence and/or frequency of cancer cells in the blood or other body fluid or tissue (e.g., a biopsy). If the tumor is impacting the normal functioning of a tissue or organ, then the effective amount may be assessed by measuring the normal functioning of the tissue or organ.

Administration may be a systemic route such as intravenous, intramuscular, intradermal, intraperitoneal, subcutaneous, by inhalation, or other parenteral routes. Administration may be oral or it may be through a localized route such as injection or topical administration to a tissue (e.g., skin, mucosa such as oral, vaginal, rectal, gut, or lung mucosa), an organ, a tumor, a lesion, a site of infection such as an abscess, and the like. The route of administration in some instances will be governed by the particular condition being treated or diagnosed.

The invention provides pharmaceutical compositions. Pharmaceutical compositions are sterile compositions that comprise nanoparticles and embedded agent(s), preferably in a pharmaceutically-acceptable carrier. The term “pharmaceutically-acceptable carrier” means one or more compatible solid or liquid filler, diluents or encapsulating substances which are suitable for administration to a human or other subject contemplated by the invention. The term “carrier” denotes an organic or inorganic ingredient, natural or synthetic, with which the cells, nanoparticles and agent(s) are combined to facilitate administration. The components of the pharmaceutical compositions are commingled in a manner that precludes interaction that would substantially impair their desired pharmaceutical efficiency.

The nanoparticle when delivered systemically may be formulated for parenteral administration by injection, e.g., by bolus injection or continuous infusion. Formulations for injection may be presented in unit dosage form, e.g., in ampoules or in multi-dose containers. Pharmaceutical parenteral formulations include aqueous solutions of the ingredients. Aqueous injection suspensions may contain substances which increase the viscosity of the suspension, such as sodium carboxymethyl cellulose, sorbitol, or dextran. Alternatively, suspensions of ingredients may be prepared as oil-based suspensions. Suitable lipophilic solvents or vehicles include fatty oils such as sesame oil, or synthetic fatty acid esters, such as ethyl oleate or triglycerides.

The following Examples are included for purposes of illustration and are not intended to limit the scope of the invention.

### Examples

We sought to develop a strategy to produce nanoparticles comprising nucleic acid gels (or crosslinked networks, and thus referred to herein interchangeably as nanoparticles or nanogels) and which encapsulate compounds regardless of size or molecular weight, and to determine the release profiles of such nanoparticles.

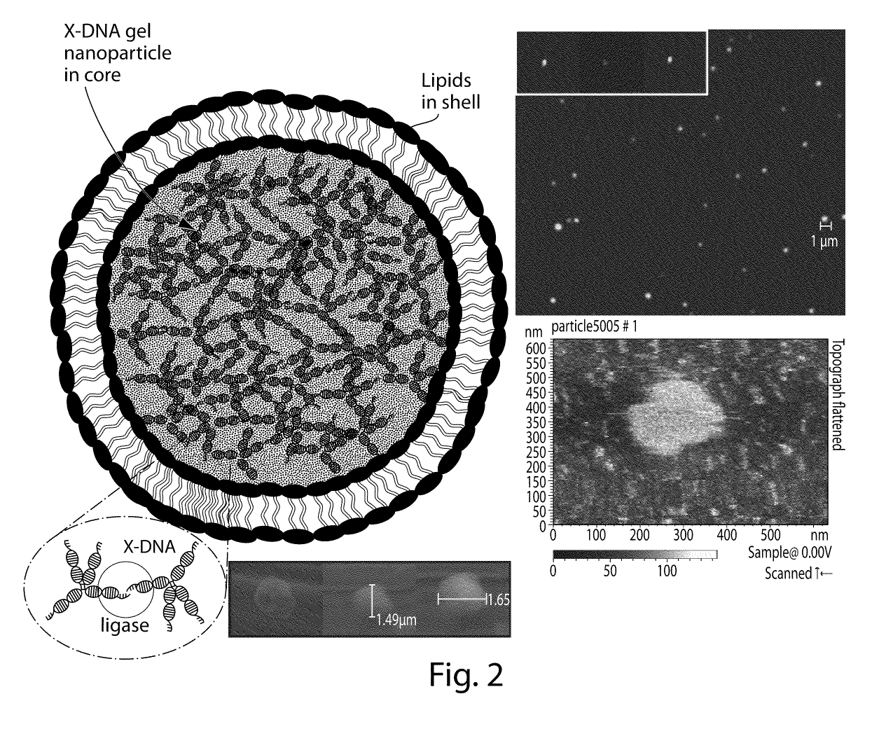

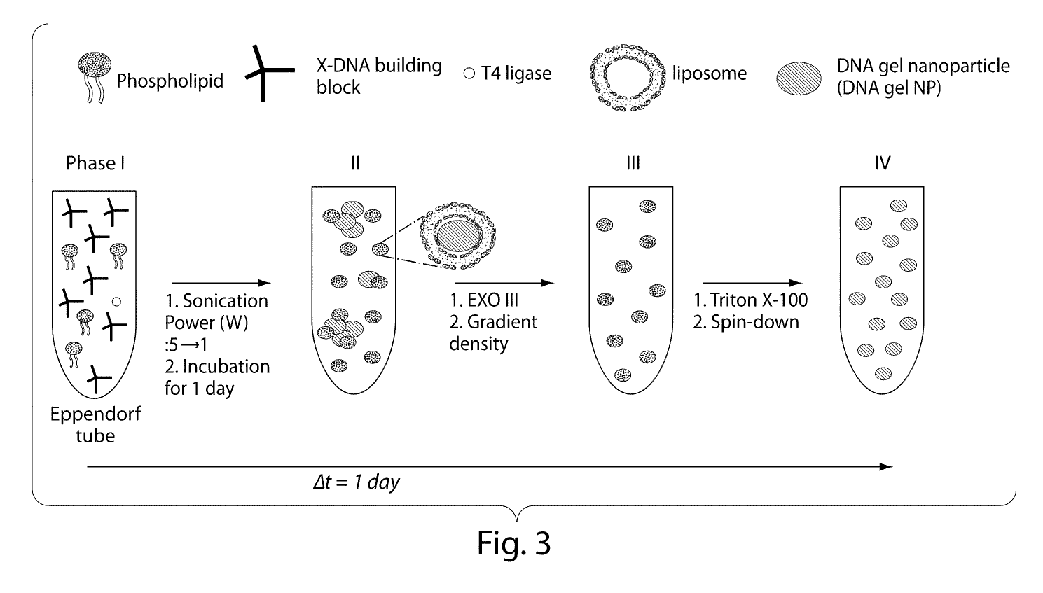

Synthesis of DNA nanogels with or without a lipid coating. The overall structure of exemplary DNA nanogels and an exemplary synthetic process (using “X-DNA” monomers) for their production is outlined in FIGS. 2 and 3, respectively. As summarized in FIG. 3, the nanogels are synthesized by a simple multistep process. First, X-DNA monomers (or building blocks), composed of 4 individual DNA strands designed to hybridize with one another into a characteristic 4-armed structure are prepared using standard molecular biology techniques. See also published US patent applications US 20070148246 A1 and US 20050130180 A1.

These DNA building blocks are then encapsulated into liposomes by rehydrating a dried phospholipid film in a vial with an aqueous solution of X-DNA and the crosslinking enzyme T4 ligase, and sonicating the lipid/DNA/enzyme mixture briefly. The size of the liposomes formed establishes the size of the resulting DNA nanogels. These liposome-like entities may then be size selected for example by passing them through membranes of reducing pore size. In this manner, populations of nanogels with a common average size can be generated. The mixture may be treated to remove free, unencapsulated nucleic acid before or after size separation and before or after crosslinking of the encapsulated nucleic acids, as discussed below.

Before or after size selection, the nanogels are incubated to covalently crosslink the ends of adjacent X-DNA arms to one another. If the crosslinking agent is T4 ligase, then a suitable incubation is 24 hrs at 16° C. (or room temperature). Other incubation times and conditions may be used, as will be apparent to those of ordinary skill in the art in accordance with the teachings herein. The resultant reaction mixture comprises crosslinked DNA gels encapsulated by lipid coatings (or liposomes) as well as “free” crosslinked DNA gel which is formed and exists outside of the liposomes (FIG. 3 Step II). Because this free DNA gel forms without a lipid “template” it does not adopt a nanogel form and instead is much larger.

Free unencapsulated DNA gel then may be degraded by treating the mixture with nuclease(s) such as exonuclease(s). The nuclease(s) targets and degrades only the unencapsulated DNA, whether or not crosslinked, while the encapsulated DNA remains intact. The mixture is finally purified by centrifugation through a sucrose density gradient to remove DNA fragments and free liposomes (FIG. 3 Step III). If lipid-free (or “naked”) DNA nanogels are desired, the purified DNA nanogels are treated to remove their lipid coating in a final step (FIG. 3 Step IV). Lipid coats may be removed using detergent such as Triton-X-100 or enzymes such as lipases and phospholipases.

FIGS. 1 and 2 schematically illustrate the final structure of DNA nanogels formed by crosslinking X-DNA monomers. X-DNA monomers are crosslinked arm-to-arm to form a 3D network within liposomal vesicles. Nanogels with sizes from ˜1 μm down to ˜100 nm diameter can be synthesized by changing the concentration of reactants and the types of lipids used in the synthesis. Also shown in FIG. 2 are confocal micrographs and a fluid-cell AFM image of DNA nanogels formed with this process. Nanogels with a range of net sizes and surface charge can be prepared with a variety of lipid coating compositions (Table 1).

TABLE 1
DNA nanoparticles with a variety of lipid components

| Sample Description | | Zeta- | Product |
|---|---|---|---|
| (e.g., DNA gel nanoparticle with the | Size | Potential | Yield |
| lipid components: DOPC X%/DOPG Y%) | (nm)* | (mV) | (%) |
| DOPC 90%/Rhod-DOPC 10% | 857 ± 28 | 0.652 | 52.0 |
| DNA nanogel alone (after lipid extraction) | 857 ± 28 | −29.04 | 48.0 |
| DOPC 90%/PEG-DSPE 10% | 100.6 ± 0.7 | 0.00147 | (very low) |
| DOPC 72%/DOPG 18%/PEG-DSPE 10% | 304.8 ± 13.2 | −3.55 | 21.6 |
| DOPC 40%/DOPG 10%/MBP-PE 50% | 797.0 ± 52.7 | 0.243 | 54.0 |

Sized by 1 micron membrane extrusion prior to crosslinking

| | | | |
|---|---|---|---|
| DOPC | 445.9 ± 24.4 | 0.223 | 58.0 |
| DOPC 40%/DOPG 10%/MBP-DOPE 50% | 310.2 ± 12.4 | −0.0268 | 52.0 |

Sized by 400 nm membrane extrusion prior to crosslinking

| | | | |
|---|---|---|---|
| DOPC 40%/DOPG 10%/MCC-DOPE 50% | 334.2 ± 4.8 | 5.39 | 48.0 |

Sized by 200 nm membrane extrusion prior to crosslinking

| | | | |
|---|---|---|---|
| DOPC 40%/DOPG 10%/MBP-DOPE 50% | 258.6 ± 6.8 | −0.027 | 54.0 |

*determined by dynamic light scattering

Lipid compositions compatible with DNA nanogel synthesis. The synthesis steps described above represent an example of an optimized synthesis scheme. It has been found according to the invention that not all lipid types can be used to prepare well-defined submicron DNA nanogels. As shown in Table 1, nanogels readily formed when zwitterionic (DOPC) and/or anionic (DOPG) phospholipids were used in the synthesis. However, addition of lipids (e.g., DSPE) conjugated to polyethylene glycol (PEG) (e.g., PEG-DSPE) reduced the yield of DNA nanogels (Table 1). Moreover, when cationic phospholipids such as DOTAP were employed in the synthesis, macroscopic DNA-lipid aggregates formed and the yield of nanogels was also very low. Thus, neutral and/or anionic lipid compositions lacking PEG headgroups appear to be optimal for synthesis of submicron DNA nanogels. If PEGylation is desired, however, it has also been determined in accordance with the invention that lipid-coated DNA nanogels are readily PEGylated post-synthesis, for example by reacting thiol-terminated PEG with maleimide-functionalized lipids used to generate the nanogels in the first instance.

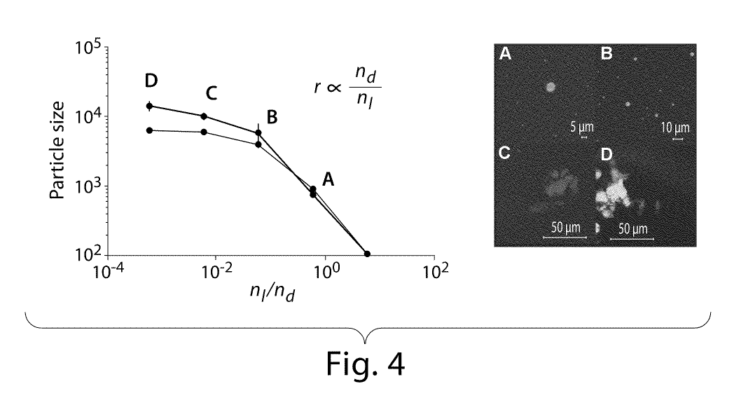

It has also been found that nanogel formation preferably occurs under certain molar ratios of X-DNA:lipid. As shown in FIG. 4 (left panel), the mean size of DNA nanogels formed in this synthesis varies with the lipid:X-DNA mole ratio (nₗ/n_{d}), with the mean particle radius (and thus also diameter) roughly inversely proportional to this ratio. Lipid:X-DNA ratios near ˜10 are suitable for generating submicron-sized nanogels. At lower ratios, macroscopic DNA-gel aggregates are formed (FIG. 4, right panel).

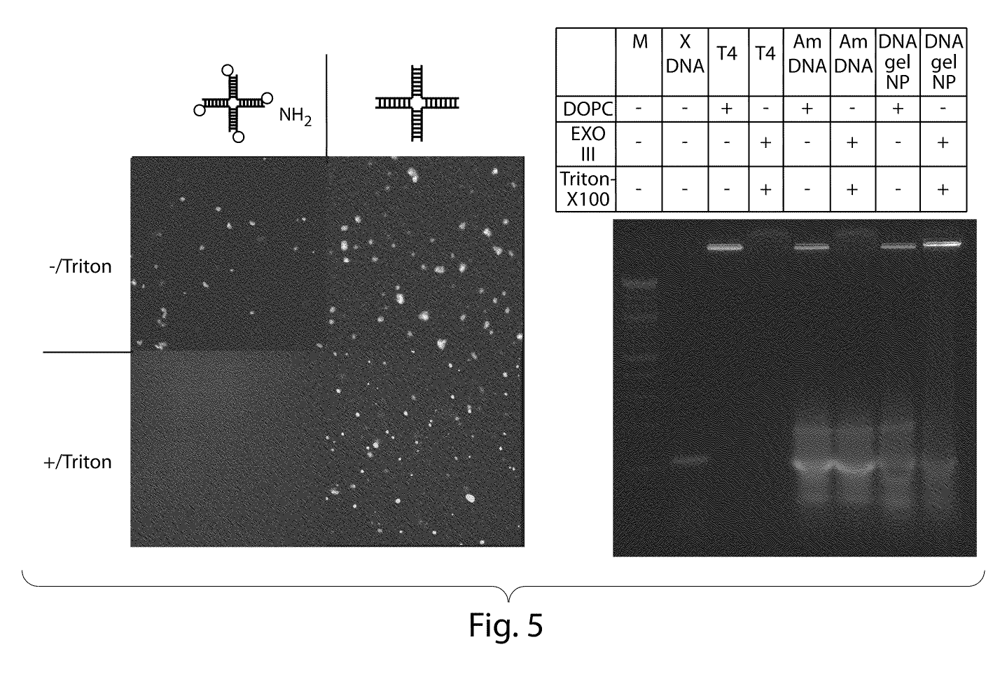

Structural analysis of DNA nanogels. The DNA nanogels were completely nontoxic to cells (data not shown). To demonstrate that the nanogels have the structure proposed in FIG. 2, DNA nanogels or control particles prepared by encapsulating within liposomes ‘dead’ X-DNA monomers end-capped with non-crosslinkable amines and T4 enzyme were analyzed. As shown in FIG. 5, particles labeled for DNA (green) and lipid (red) show the components colocalized in punctate spots following particle synthesis. However, DNA nanogels prepared using crosslinkable X-DNA monomers treated with the detergent Triton X-100 were stable, while the control uncrosslinked X-DNAs dispersed once the encapsulating lipid bilayer was removed. Similarly, if the synthesis was carried out in the absence of T4 ligase, then lipid encapsulated X-DNA was obtained but it dissolved following Triton-X treatment to disrupt the lipid coating. These results prove that enzyme-mediated crosslinking is occurring and is required for the formation of stable DNA nanogels.

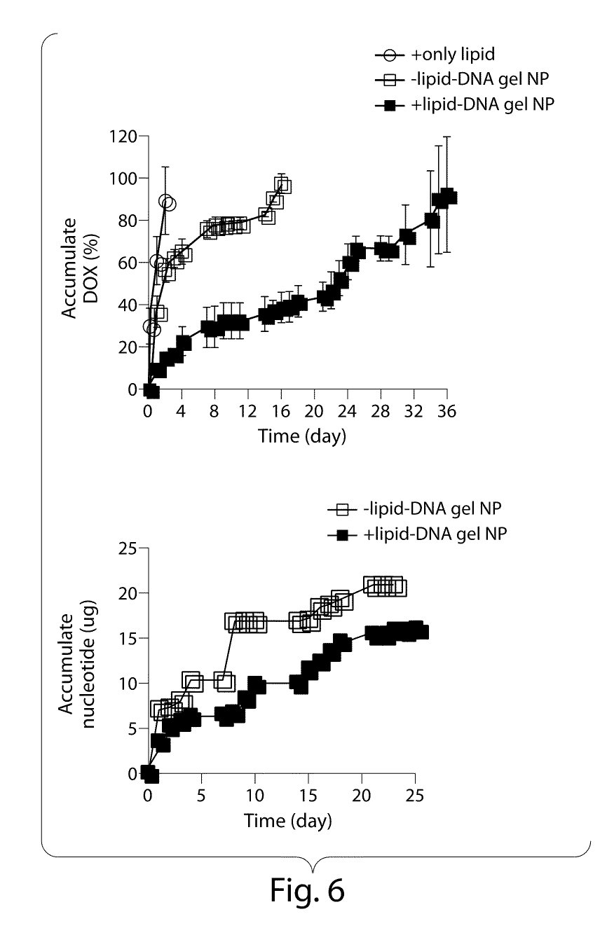

Using DNA nanogels to bind and slowly release a chemotherapy drug. Doxorubicin (dox, DOXIL) is a well-known chemotherapy agent that is a component of standard treatment for several cancers. Doxorubicin use is limited by cardiac toxicity, and an FDA-approved STEALTH liposome formulation of doxorubicin is currently used to allow higher doses of doxorubicin to be delivered by lowering cardiac exposure and elevating intratumoral drug accumulation. Because doxorubicin binds with high affinity to double-stranded DNA as part of its mode of action, we tested whether DNA nanogels could load high amounts of doxorubicin by binding the drug to the double stranded regions of the gel, and slowly release the drug over time. As shown in FIG. 6, doxorubicin was efficiently loaded to high levels in lipid-coated or uncoated (“naked”) DNA nanogels. Lipid-coated DNA nanogels loaded ˜110 μg doxorubicin per μg of lipid, compared to ˜23 μg doxorubicin per μg lipid for standard liposomes. We encapsulated doxorubicin in liposomes, lipid-coated DNA nanogels, or uncoated DNA nanogels, and measured the release of the drug into serum-containing medium at 37° C. over time via spectrofluorimetry. Regular liposomes, known to be unstable in serum, released their entire doxorubicin content within 3 days. In contrast, naked doxorubicin-loaded DNA nanogels released doxorubicin for ˜2 weeks, and lipid-coated DNA nanogels showed even more prolonged release, continuing to release the drug for ˜1 month. Thus, the DNA structure of these gels can be used to greatly prolong the controlled release of DNA-binding chemotherapy agents, among others.

This experiment also demonstrated the high stability of crosslinked X-DNA structures loaded with doxorubicin, even in the presence of serum DNases. When the cumulative release of nucleotide fragments from the nanogels was simultaneously tracked in this experiment, we found that nanogels were slowly degraded over a period of 20 days to more than one month for uncoated and lipid-coated nanogels, respectively (FIG. 6, right panel).

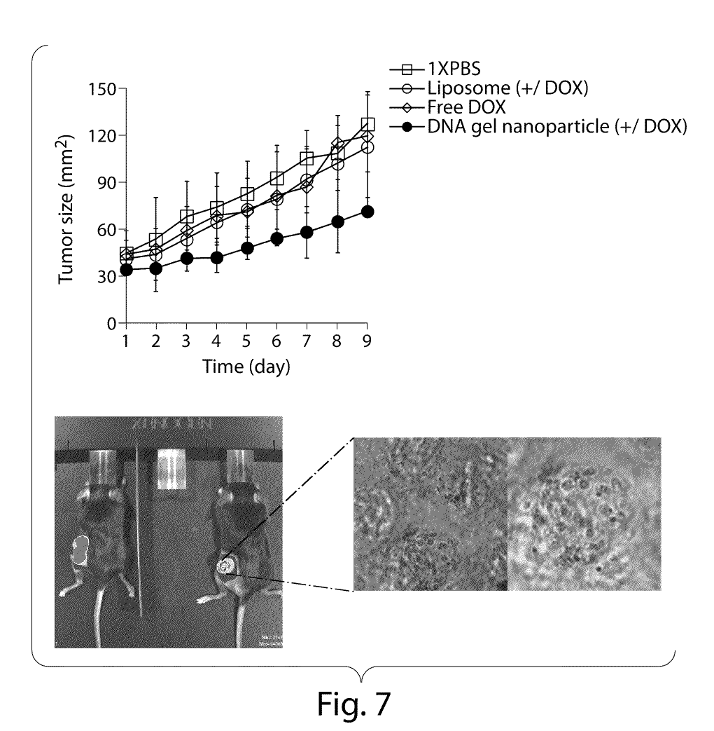

To test whether doxorubicin-loaded DNA nanogels could provide a sustained therapeutic effect in vivo, we treated large established B16F10 melanoma tumors in groups of immunocompetent C57B1/6 mice with a single injection of free doxorubicin, doxorubicin encapsulated in liposomes, or doxorubicin encapsulated in lipid-coated DNA nanogels. Control animals received injections of saline alone. As shown in FIG. 7, a single intratumoral injection of free doxorubicin or doxorubicin-encapsulated in liposomes had no effect on tumor growth relative to saline-injected control animals, while doxorubicin-loaded DNA nanogels slowed tumor growth for at least a week following a single injection.

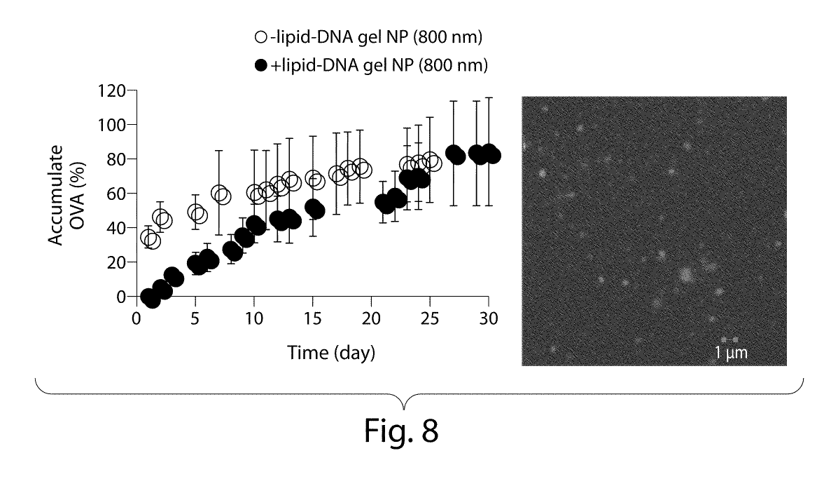

Entrapment and sustained release of proteins. DNA nanogels can also entrap macromolecules within their X-DNA meshwork, for slow release by diffusion and/or slow degradation of the DNA gel structure in the presence of serum. We entrapped fluorescent ovalbumin (OVA, 46 KDa globular protein) within DNA nanogels by adding OVA to the X-DNA monomer/enzyme solution during Step I of the synthesis, achieving encapsulation levels of ˜12 μg OVA per mg of DNA. Such encapsulation levels are comparable to that typically achieved by encapsulation in biodegradable polymer particles. However, here no organic solvents are used in the process, and thus more of the protein activity should remain after synthesis. Release of OVA from lipid-coated or uncoated DNA nanogels into serum-containing medium at 37° C. was then tracked over 4 weeks. As shown in FIG. 8, protein was released from the DNA nanogels (˜800 nm diameter) over a period of 25-30 days with or without the lipid surface coating. Such prolonged, sustained release from nanogels is typically difficult to achieve using prior art methods.

### REFERENCES

1. Urn, S. H., Lee, J. B., Park, N., Kwon, S. Y., Umbach, C. C., and Luo, D., Enzyme-catalysed assembly of DNA hydrogel. Nat Mater 5 (10), 797 (2006).
2. Um, S. H., Lee, J. B., Kwon, S. Y., Li, Y., and Luo, D., Dendrimer-like DNA-based fluorescence nanobarcodes. Nat Protoc 1 (2), 995 (2006).
3. Park, N., Um, S. H., Funabashi, H., Xu, J., and Luo, D., A cell-free protein-producing gel. Nat Mater (2009).

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-37. (canceled)

38. A submicron-sized particle of crosslinked nucleic acids made according to a method comprising
combining in solution branched nucleic acids, nucleic acid ligase, ATP, and lipids to form a mixture comprising lipid-encapsulated branched nucleic acids and free branched nucleic acids,
incubating the mixture under conditions and for a time sufficient for the nucleic acid ligase to crosslink the branched nucleic acids, and
harvesting crosslinked branched nucleic acids.

39-41. (canceled)

42. A particle comprising crosslinked branched nucleic acids and having an average diameter in the range of about 100 nm to about 1 micron.

43-81. (canceled)

82. A method comprising
administering the particle of claim 38 to a subject in need thereof in an effective amount.

83-95. (canceled)

96. A method comprising releasing or maintaining an agent in a subject for a period of about 2-4 weeks following administration to a subject of a particle comprising the agent, wherein the particle is the particle of claim 38.

97. A method comprising
administering the particle of claim 42 to a subject in need thereof in an effective amount.

98. A method comprising
releasing or maintaining an agent in a subject for a period of about 2-4 weeks following administration to a subject of a particle comprising the agent, wherein the particle is the particle of claim 42.

99. The particle of claim 38, wherein the lipids are non-cationic phospholipids.

100. The particle of claim 38, wherein the method further comprises removing the free branched nucleic acids from the mixture.

101. The particle of claim 38, wherein the harvested crosslinked branched nucleic acids are lipid-encapsulated.

102. The particle of claim 38, wherein the method further comprises removing lipids from the mixture prior to harvesting crosslinked branched nucleic acids.

103. The particle of claim 38, wherein the harvested crosslinked branched nucleic acids do not have a lipid coating.

104. The particle of claim 38, wherein the method further comprises size selecting the lipid-encapsulated branched nucleic acids.

105. The particle of claim 38, wherein the method further comprises size selecting the crosslinked branched nucleic acids before or after harvest.

106. The particle of claim 100, wherein the free branched nucleic acids are removed from the mixture using a nuclease.

107. The particle of claim 106, wherein the nuclease is exonuclease.

108. The particle of claim 102, wherein the lipids are removed using detergent or an enzyme.

109. The particle of claim 108, wherein the detergent is Triton-X and the enzyme is a lipase.

110. The particle of claim 38, wherein the branched nucleic acids are branched DNA.

111. The particle of claim 38, wherein the branched nucleic acids comprise a therapeutic agent.
